(19) United States
(12) Patent Application Publication
(10) Pub. No.: US 2006/0173276 A1
(43) Pub. Date: Aug. 3, 2006

(54) Contrast-enhanced moving table mr arteriography with stochastic sampling of central k-space

(76) Inventors: Johan Samuel Van Den Brink, Eindhoven (NL); Miha Fuderer, Eindhoven (NL); Paul Royston Harvey, Eindhoven (NL)

Correspondence Address: PHILIPS INTELLECTUAL PROPERTY & STANDARDS, 595 MINER ROAD, CLEVELAND, OH, 44143, US

(21) Appl. No.: 10/559,160

(22) PCT Filed: May 26, 2004
(86) PCT No.: PCT/IB04/50779

§ 371 Date: Dec. 2, 2005

(30) Foreign Application Priority Data
Jun. 3, 2003 (EP) 03101615.7

(51) Int. Cl. A61B 5/05

(52) U.S. Cl. 600/410

(57) ABSTRACT

A system is described for the acquisition of a magnetic resonance scan of a subject. The system is particularly useful when the subject which contains a volume of interest which is larger than the field of view of the system and when the scan includes a time dependent signal. The system also comprising a subject support capable of movement relative to the field of view. The system presented is arranged to perform at least a first scan and a second scan, the first scan being arranged so that signal is acquired from the central region of k-space as the subject support is moved through the field of view in a first direction and the second scan being arranged so that signal is acquired from the periphery of k-space as the subject support is moved through the field of view in a second direction.

The invention relates to a system for the acquisition of a magnetic resonance scan of a subject which contains a volume of interest, which scan includes a time dependent signal, the system comprising a field of view which is smaller than the volume of interest and also comprising a subject support capable of movement relative to the field of view.

Such a system is described in WO 01/73462 which discloses a magnetic resonance imaging system and method for performing a magnetic resonance contrast scan in which k-space is partitioned into at least two sectors which are then used to form the basis of separate and distinct acquisitions of magnetic resonance signal. In the described system in its most basic form, k-space is partitioned into at least a central portion and a peripheral portion. An allowance is made for the possibility of the existence of an intermediate portion between the central and peripheral portions. This division of k-space separates the acquisition of the image signal information into contrast and resolution information and solves the problem of how to acquire sufficient image data during a time dependent contrast scan.

The time dependent contrast scan disclosed in WO 01/73462 is an arterial contrast scan in which a contrast medium flowing in the blood circulatory system of the subject is required to be imaged while it still resides in the arterial system. The contrast agent utilized produces a strong signal when it interacts with the magnetic and electromagnetic fields of the system with the result that any vessels in which it is contained show up against the rest of the tissue. This allows visual examination of the vessels and provides useful information regarding the patency of the arteries while the agent remains in the arteries. Once the contrast agent has moved into the venous system the resulting images are contaminated with contrast signal originating from the veins. Maximally effective images are produced while the contrast agent remains within the arterial system and this imposes a time constraint on the overall imaging procedure which is solved by the disclosed method. The solution is to acquire signal from the center of k-space providing contrast information, during an initial phase of the image acquisition. This phase continues until the contrast agent has started to transfer into the venous system at which point signal from the periphery of k-space, describing resolution information is acquired. Thus the acquisition of contrast information is maximized while the contrast agent is still in the arterial bed and the acquisition of resolution information is only acquired when the contrast agent has moved into the venous bed.

There is a current move within magnetic resonance imaging towards the use of smaller magnets. In part this move is driven by the realization that substantial numbers of patients are uncomfortable and sometimes claustrophobic inside the long, enclosed bores of traditional magnetic resonance imagers. A shorter bore magnet is currently used as the solution to this problem and in addition has the advantages that it is cheaper to produce and takes up less space. However, such a magnet has a correspondingly smaller field of view and therefore brings with it an entirely different set of problems, for instance those associated with the acquisition of magnetic resonance signal from a volume of interest larger than the field of view of the imager. A solution to this problem is the moving bed. Subjects for study in the magnetic resonance imager are placed on a patient support, or bed, to be moved into and out of the imager. Traditionally, the patient was positioned inside the bore of the magnet and was required to remain lying still while the full acquisition was completed. But the solution of the moving bed allows the subject to be moved across the field of view of the magnet while the imaging sequence acquires signal from the whichever section of the volume of interest is inside the bore of the magnet and available for study. Therefore a whole volume of interest can be interrogated for imaging signal over a period of time. The patient is effectively moved in a step-wise fashion through the field of view of the magnet and with each step, magnetic resonance signal is acquired from a different sub-volume of the overall volume of interest. The patient remains stationary within the bore of the magnet within each step, so that the effect of the moving bed is to break down the overall acquisition from a larger stationary volume into smaller stationary sub-sections. The sub-volumes of acquired image data are concatenated to form the overall image volume representing the entire volume of study.

However, this method and the moving bed technology to which it is applied is not applicable to time dependent scans, for example an arterial contrast scan. The method could be performed over any one of the sub-volumes that make up the overall image once the moving bed had moved the subject to the correct position, but by the time both phases of the scan had been acquired the contrast agent would have moved into the venous system of the patient and out of the arterial bed. The subsequent acquisitions from the other sub-volumes would therefore be rendered useless.

It is an object of the invention to provide a scanning procedure which can be applied to a magnet with a field of view which is smaller than the volume of interest in the subject for study and which still allows the acquisition of a fully time dependent scan over a limited period of time.

This is realized according to the invention in which the system is arranged to perform a first scan in which signal is acquired from the central region of k-space as the subject support is moved through the field of view in a first direction and the system is arranged to perform a second scan in which signal is acquired from the periphery of k-space as the subject support is moved through the field of view in a second direction which is preferably opposite to the first direction.

Claim 1 defines a system for the acquisition of a magnetic resonance scan using a moving bed in which the bed moves the subject for study into and out of the field of view of the imager. Such a system usually comprises a main magnetic field which is so arranged to be permanently activated with, in addition, a series of gradient coils and radiofrequency coils arranged to produce the gradient fields and radiofrequency fields needed to select and extract magnetic resonance signal from the volume of interest. The volume of interest is the area within the subject which is the focus of study and of which it is intended to produce an image. The image is acquired, or extracted, using magnetic resonance sequences, that is, ordered combinations of varying magnetic fields which produce and detect varying signals from the protons within the subject. The magnetic resonance signal is frequently extracted in lines of signal data which can be built up into a full data set corresponding to the real space taken up by the volume of interest.

Systems designed to acquire magnetic resonance scans form one part of a group of imagers sometimes referred to as volume imagers, because the data sets which they produce represent whole volumes of tissue. The data sets can be viewed in a number of ways to get the most out of the volume information. For example, they can be viewed as 2 dimensional slices of image data orientated at any angle as in the commonly used technique of multi-planar reformatting, or, they can be viewed as volume displays using surface and volume rendering techniques to render entire portions of the volume display. Pixels or voxels within the images are commonly reproduced in a range of intensities to represent the strength of the signal from different parts of the image.

The information held within an image can be decomposed into monowavelength signals which describe individual frequency components of the overall image. In general, high frequency signals represent visual information with a high resolution, such as information describing finely detailed structures or physical edges and tissue boundaries. Low frequency signals generally convey low resolution information which includes broad structural image content. In order to contain enough clinically useful information, that is gross anatomical information at a sufficiently high resolution, a typical medical image is constructed from a wide range of visual frequencies.

The Fourier transform of an image is known as k-space and the central region of k-space represents the low wave number, long wavelength components of the image. As such, this region of k-space contains information describing the gross anatomy within the image. The outer, or peripheral, regions of k-space represent the high wave number, short wavelength components of the image. This region of k-space therefore encodes the high resolution information contained in the image. Acquisition of complete image requires acquisition of image signal over the complete region of k-space.

This acquisition of the full set of signals required to complete the multifrequency description of the image requires repeated scanning of the volume of interest using different gradient fields. This requires time for acquisition at different gradient field combinations and imposes a time constraint on any system in which acquisition must also be effected using a shortened magnetic bore.

The system defined in claim 1 solves this problem by dividing up the overall acquisition into two distinct temporal sections. A first section in which the subject for study is moved into the field of view of the system during which time image data from the center of k-space is acquired, and a second section in which the subject for study is moved out of the field of view of the system during which time data from the periphery of k-space is acquired. This has the effect of allowing a full set of information to be acquired from the subject while remaining within the time constraints imposed by the scan.

In practice, a volume of interest is defined within the body of the subject. For a contrast arterial scan this would be a volume incorporating part of the total arterial tree. For example it could include the heart, the aorta and major arteries proceeding down to the lower limbs. Alternatively it might include the heart, ascending aorta, brachial arteries and carotids. The subject, lying still on the subject support, is then moved, via the subject support, through the field of view of the imager while signal is acquired. The movement of the subject support allows portions of the volume of interest to be present in the field of view of the imager at some point during the signal acquisition so that signal is derived from relevant extents of the volume of interest. At the end of this first phase of movement, the subject support is stopped and a second phase of movement is initiated. This second phase of movement is in a direction opposite to the original direction of movement of the patient support. This reversal of direction of the moving bed allows the signal in the second phase of the acquisition to be acquired from the same volume of interest. In this way, the complete amount of signal is acquired to build up a full MR image.

The division of the acquisition of signal from k-space into two separate portions divides the acquisition of the overall image information into an acquisition of low resolution information and a separate acquisition of high resolution information, with the added feature that the two separate portions of information are acquired from the entire volume of interest during the same overall acquisition. That is to say, the inherent limitation of the stepping movement of the moving bed, and the attendant division of the volume of interest into sub-volumes, is avoided. The method thereby allows the acquisition of the total signal, but over a volume of interest which is greater than the field of view of the main magnetic field.

The acquisition of the signal can be acquired using any method for scanning k-space. Claim 2 discloses that the signal acquired from the center of k-space is acquired stochastically, that is to say, randomly. This has the advantages that artefacts are reduced.

Claim 3 discloses the arrangement of the relative speeds of the two separate phases of the acquisition. Because the intention is to collect contrast information while the contrast agent remains in the portion of the subject anatomy which is of interest, the speed of the moving bed should be set so that the volume of interest in the subject passes through the field of view during the first phase of the scan in the time for which the contrast agent resides in the anatomy of interest. Typically, this can be achieved using a velocity of 5 centimeters per second. Once this first phase of the scan has been completed the subject is moved back out again at a second speed. This speed is generally slower than the first speed because there is no longer any time pressure on the acquisition of the information contained in the high k-value signal. A velocity of 0.5 centimeters per second has found to be advantageous during this second phase.

The system disclosed is particularly applicable to contrast enhanced angiography. In this technique of magnetic resonance imaging, enhanced contrast is arranged within the circulatory system in order to render the anatomy of the circulatory vessels visible within the constraints imposed by the magnetic resonance method. This enhanced contrast is usually provided by the use of a contrast agent introduced into the circulatory system of the subject. The contrast agent produces a strong magnetic resonance signal and is usually introduced into the subject for study via a vein which transports the contrast agent to the heart and thence out into the arterial tree. From there it continues under arterial pressure into the capillary bed and into the venous system.

This contrast agent, contained within the arteries, provides a sufficiently strong signal emanating from the lumina, or internal volume, of the circulatory vessels for these to appear in the final image with sufficient resolution for individual anatomy and irregularities to be seen. The signal thus allows visualization of the network of arterial spaces available for the transport of blood and as such allows the visual identification of abnormalities and pathology within this arterial network. Such abnormalities may be physical abnormalities such as dissections, but are also likely to be pathological irregularities such as narrowings, or stenoses, of the vessel lumina.

Once the contrast agent has started to transfer out of the arterial bed, however, and into the capillaries, the likelihood of the arteries providing a useful signal decreases. At the same time, contrast medium starts to appear in the veins and so any subsequent image data will be contaminated by venous signal. A magnetic resonance scan of the arterial contrast agent must therefore be acquired while the contrast agent still resides in the arterial tree and before it has moved into the venous bed. It is this fact which provides the time constraint on the acquisition of the signal.

The technique allows the acquisition of useful signal from the subject for imaging by allowing the acquisition of low resolution information conveying bulk contrast information at the start of the scan while the contrast agent is still within the arterial bed and before it has transferred appreciably into the venous bed. This is the signal which is collected while the subject is moved initially into the field of view of the system. The remainder of the signal, that which provides higher resolution detail, is then acquired when the contrast agent has moved out of the arterial bed, and useful image information about the state of the contrast agent can no longer be acquired. This second phase of the scan is the phase which is undergone while the subject is moved out of the field of view of the imager. In both phases, it is the same overall volume of interest in the subject which is interrogated for magnetic resonance signal. The difference is that image data describing the bulk contrast in the image is acquired in the first phase, while the contrast agent is still in the arteries while data describing the fine detail in the image is acquired only in the second phase, when the contrast agent has moved out of the arteries and into the veins.

The invention also relates to a method of acquiring a magnetic resonance scan of a subject containing a volume of interest, in which the volume of interest is larger than an imaging volume available to acquire the magnetic resonance signal, which magnetic resonance scan also contains a time dependent signal, and involves the use of a subject support capable of movement relative to the imaging volume.

It is an object of the invention to provide a method which allows the acquisition of magnetic resonance signals according to a scanning procedure which can be applied to a magnet with a field of view which is smaller than the volume of interest in the subject for study and which still allows the acquisition of a fully time dependent scan over a limited period of time.

This is realized according to the method of the invention in which a first scan is performed in which signal is acquired from the central region of k-space as the subject support is moved through the imaging volume in a first direction, and a second scan is performed in which signal is acquired from the periphery of k-space as the subject support is moved through the imaging volume in a second direction which is preferably opposite to the first direction.

The invention also relates to a computer program arranged to acquire a magnetic resonance signal from an imaging volume, which signal can be used to form a magnetic resonance image of a subject.

It is an object of the invention to provide a computer program capable of performing the above invention.

This is realized according to the method of the invention in which the computer program contains instructions to acquire a magnetic resonance signal from the central region of k-space as the subject is moved through the imaging volume in a first direction, it also contains instructions to acquire a magnetic resonance signal from the periphery of k-space as the subject is moved through the imaging volume in a second direction which is preferably opposite to the first direction.

These and other aspects of the invention will be further described using the following figures.

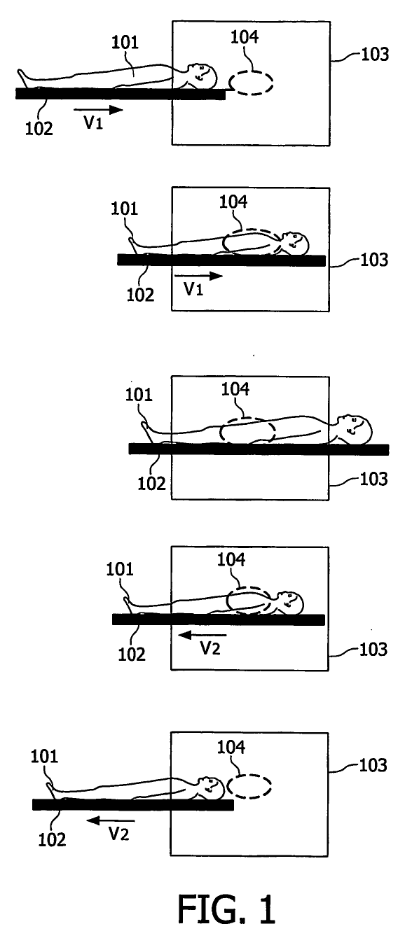

FIG. 1 shows the movement of the subject support into and out of the magnetic resonance system according to the invention.

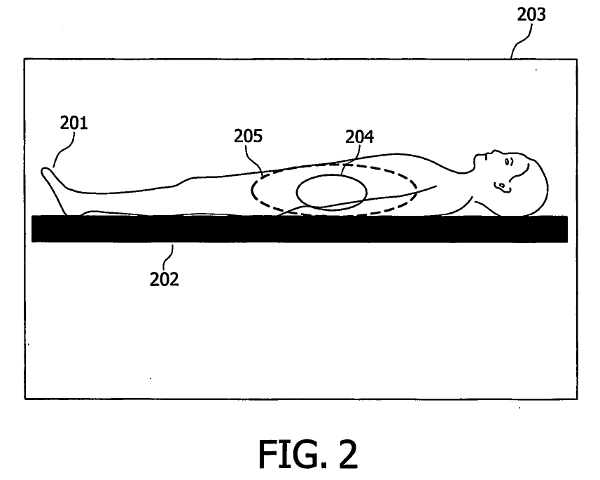

FIG. 2 shows the relationship between the volume of interest in the subject for study and the field of view of the magnetic resonance system in a clinical imaging situation in which the invention would be advantageous.

FIG. 1 shows a sequence of events in which a subject for study 101 moves on a subject support, or bed, 102 into a magnetic resonance system, or imager, 103 containing a field of view 104. The subject support moves into the magnetic resonance system at a first speed v1 and moves out again at a second speed v2.

FIG. 2 shows the volume of interest 205 of the subject for study 201, itself situated on the subject support 202 within the magnetic resonance system 203. The size differential between the volume of interest 205 and the smaller field of view 204 of the magnet is shown. It is clear from the figure that the volume of interest within the patient is too large to be accommodated within the field of view of the magnet.

As can be seen from the above explanations, the aim of the method is to acquire a full data set of information allowing reconstruction of a volume image covering the full anatomical volume of interest. However, as will be appreciated, image information is acquired from the subject while the subject is moving relative to the field of view. The acquisition can be achieved by shifting the physical origin of the acquired image data to maintain the correct relative position to the volume of interest in the subject. Signal received during the scanning phases of the systems is acquired in lines. These lines of data are allocated to the overall 3 dimensional data set by allocating them to their correct position in the 3 dimensional k-space. It is common in volume imaging to set up the acquisition sequence so that the information signal is read out in a direction perpendicular to the main axis of the direction of movement of the moving bed and parallel to a coronal plane of the subject. The lines of data thus acquired are allocated to the resulting k-space data set using knowledge about the position of the subject support. The final 3 dimensional image is produced from the Fourier transform of the completed k-space data set

The magnetic resonance system is depicted within the figures as a closed system. It will be appreciated by one skilled in the art that the same references and comments can be made to an open magnet system.

1. A system for the acquisition of a magnetic resonance scan of a subject which contains a volume of interest, which scan includes a time dependent signal, the system comprising a field of view which is smaller than the volume of interest and also comprising a subject support capable of movement relative to the field of view, wherein,
the system is arranged to perform at least a first scan and a second scan,
the first scan is arranged so that signal is acquired from the central region of k-space as the subject support is moved through the field of view in a first direction,
the second scan is arranged so that signal is acquired from the periphery of k-space as the subject support is moved through the field of view in a second direction which is preferably opposite to the first direction.

2. A system as claimed in claim 1, wherein, the system is arranged such that the signal which is acquired from the central region of k space is acquired stochastically.

3. A system as claimed in claim 1, wherein,
the system is arranged such that the subject support is moved into the field of view at a first speed,
the system is arranged such that the subject support is moved out of the field of view at a second speed,
the first speed is faster than the second speed.

4. A system as claimed in claim 1, wherein, the subject support is moved with a speed which is variable.

5. A system as claimed in claim 1, wherein the time dependent signal includes signal information which originates from a contrast medium.

6. A method of acquiring a magnetic resonance scan of a subject containing a volume of interest, in which the volume of interest is larger than the field of view available to acquire the magnetic resonance signal, which magnetic resonance scan also contains a time dependent signal, and involves the use of a subject support capable of movement relative to the field of view, wherein,
the system is arranged to perform at least a first scan and a second scan,
the first scan is arranged so that signal is acquired from the central region of k-space as the subject support is moved through the field of view in a first direction,
the second scan is arranged so that signal is acquired from the periphery of k-space as the subject support is moved through the field of view in a second direction which is preferably opposite to the first direction.

7. A computer program arranged to acquire a magnetic resonance signal from an imaging volume, which signal can be used to form a magnetic resonance image of a subject, wherein,
it contains instructions to acquire a magnetic resonance signal from the central region of k-space as the subject is moved through the imaging volume in a first direction,
it also contains instructions to acquire a magnetic resonance signal from the periphery of k-space as the subject is moved through the imaging volume in a second direction which is preferably opposite to the first direction.
